(19) United States
(12) Patent Application Publication
(10) Pub. No.: US 2025/0313596 A1
(43) Pub. Date: Oct. 9, 2025

(54) BACILLUS SUBTILIS STRAINS PRODUCING NEW MYCOSUBTILIN ISOFORMS

(71) Applicants: Universite de Lille, Lille (FR); Lipofabrik, Lesquin (FR)

(72) Inventors: François Coutte, Lille (FR); Arnaud Delecroix, Lesquin (FR); Debarun Dhali, Lesquin (FR); Philippe Jacques, Lesquin (FR); Tambi Kar, Lesquin (FR); Yazen Yaseen, Lesquin (FR)

(21) Appl. No.: 19/222,344

(22) Filed: May 29, 2025

Related U.S. Application Data
(63) Continuation-in-part of application No. PCT/EP2023/083606, filed on Nov. 29, 2023.

(30) Foreign Application Priority Data
Nov. 29, 2022 (FR) 2212465

(51) Int. Cl. C07K 7/64; C12N 15/75; C12P 21/02

(52) U.S. Cl. CPC C07K 7/64; C12N 15/75; C12P 21/02; C12N 2800/101

(57) ABSTRACT

The present disclosure relates to the field of molecules of antifungal biosurfactants of bacterial origin. More particularly, the present disclosure relates to new Bacillus Subtilis strains producing new isoforms of mycosubtilins, a preparation method therefor, and compositions containing same. These novel mycosubtilin isoforms exhibit improved antifungal activities and reduced cytotoxic properties compared with mycosubtilins of the prior art.

### CROSS-REFERENCE TO RELATED APPLICATIONS

Pursuant to 35 U.S.C. § 111(a), this application is a continuation-in-part of International Patent Application PCT/EP2023/083606, filed Nov. 29, 2023, which claims the benefit of French Patent Application Serial No. FR2212465, filed Nov. 29, 2022, the disclosure of each of which is hereby incorporated herein in its entirety by this reference.

### STATEMENT REGARDING ELECTRONIC FILING OF A SEQUENCE LISTING

Pursuant to 37 C.F.R. § 1.831 through 1.835, a Sequence Listing XML file entitled “Sequence Listing-18388US.xml,” 45 KB in size, generated Nov. 29, 2023, has been submitted via EFS-Web is provided in lieu of a paper copy. This Sequence Listing is hereby incorporated by reference into the specification for its disclosures.

### TECHNICAL FIELD

The present disclosure relates to the field of molecules of antifungal biosurfactants of bacterial origin. More particularly, the present disclosure relates to new Bacillus subtilis strains producing new isoforms of mycosubtilins, a preparation method therefor, and compositions containing same. These novel mycosubtilin isoforms exhibit improved antifungal activities and reduced cytotoxic properties compared with mycosubtilins of the prior art.

### BACKGROUND

The world's population is growing, and is expected to reach 8.3 billion in 2025 and almost 10 billion in 2050. Depending on the different assumptions used, global food demand could increase by between 40% and 68% by 2050 (Hérault, 2011). This increase in demand could be met by even more intensive agriculture, involving higher consumption of water, fossil fuels, fertilizers and plant protection products to ensure high yields. To meet these challenges, the scientific community and industry must develop innovative research approaches. In the field of agriculture, this raises the issue of securing yields and farm profitability, while limiting the use of inputs derived from chemical synthesis. The latter are the subject of much debate, and their impact on the health of farmers, consumers, the environment and biodiversity has been demonstrated in numerous cases. Authorities around the world have introduced regulations to limit the use of toxic compounds in the agricultural, food and other sectors.

Demand for organic molecules in agriculture is therefore very high. A recent report indicates that the biopesticides market is set to reach 8.5 billion USD by 2025. Other business sectors are also heavily impacted by increased regulatory pressure on synthetic molecules in Europe since the implementation of the REACH directive in 2007. In particular, manufacturers are looking for preservatives, biosurfactants and new antimicrobial molecules. Microbial secondary metabolites produced by numerous micro-organisms such as B. subtilis possess all these properties.

In the agricultural sector, there are many plant pathogens (around 7,000 species), and among them, Botrytis cinerea (responsible for gray mold) and Zymoseptoria tritici (responsible for wheat septoria) rank in the top 10 plant pathogens with the greatest impact on crops (Dean et al. 2012).

Lipopeptides are bioactive molecules produced by various bacterial strains, notably Bacillus and Pseudomonas (Jacques, 2011). Bacillus strains are capable of producing three different families of molecules: surfactins (surfactin, pumillacidin); fengycins (fengycin, plipastatin, agrastatin); and iturins (iturin, mycosubtilin, bacilomycin, mojavensin). Among the iturin family, mycosubtilin is the compound with the highest antifungal activity (Besson et al., 1979). Mycosubtilin is a lipopeptide whose heptapeptide ring is linked to a fatty acid chain by a [β-amino] bond. The length of the fatty acid chain can vary from C15 to C18, and the isomerism can be linear, iso or anteiso. (Stein, 2005). The classic peptide chain formula is Asn/D-Tyr/D-Asn/Gln/Pro//D-Ser/Asn, as disclosed in FIG. 1.

The composition of the peptide ring, as well as the length and isomerism of the fatty acid chain, are factors that influence the molecule's activity, particularly its antifungal activity. In 2009, a study showed that the anteiso-C17 isoform of mycosubtilin was the most active against the yeast C. albicans. (Fickers et al., 2009). In 2013, another work studied the effect of different alkyl chains of mycosubtilin against B. cinerea. The authors showed that the anteiso-C17 isoform was the most active against B. cinerea with a MIC of 8 μM, followed by the n-C16 and iso-C17 (MIC=16 μM) and iso-C16 (MIC=32 μM) isoforms. Furthermore, the presence (even at low concentration) of the C18 chain was shown to significantly enhance the activity of the mixture, suggesting a very potent activity for this isoform (Béchet et al., 2013). More recently, the antifungal activities of mycosubtilin isoforms have been measured against A. niger, the results showing that the anteiso-C17 isoform was the most active against A. niger with a MIC of 8 μM, followed by the n-C16 and iso-C17 isoforms (MIC=16 μM) and iso-C16 (MIC=32 μM). (J.-S. Guez et al., 2022).

Numerous scientific studies have demonstrated the broad spectrum of mycosubtilin's anti-fungal activity. This is the case against Candida albicans, Bremia lactucae, Zymoseptoria tritici, Botrytis cinera, Fusarium oxysporum, Fusarium graminearum and Fusarium verticillioides Candida krusei, Paecilomyces variotii, Byssocchlamys fulva, Venturia inaequalis, Verticillium dahlia or Aspergillus niger (Béchet et al., 2013; Chen et al., 2021; Deravel et al., 2014; Desmyttere et al., 2019; Farace et al., 2015; Fickers et al., 2009; Guez et al., 2022; Kourmentza et al., 2021; Mejri et al., 2017; Mihalache et al., 2018; Yu et al., 2021).

In 2011, a new mycosubtilin isoform was discovered in a modified strain of B. subtilis and patented. This molecule with a C17 fatty acid chain contains a Glutamine in position 3 [Gln3] instead of an Asparagine (WO 2013/050700). In this patent, it was shown that a mixture of mycosubtilin isoforms containing this new isoform [Gln3-C17] (even in small quantities, of the order of 1%) was as active as a mixture not containing it, demonstrating the better antifungal power of this isoform with respect to the others.

There are very few studies on the toxicity and ecotoxicity of the lipopeptide molecules produced by B. subtilis and, in particular, of each lipopeptide isoform. Ecotoxicity was studied on mixtures of surfactin and mycosubtilin isoforms alone or in binary mixtures. The results of this study showed that the EC50 of mycosubtilins in the microtox test (on Aliivibrio fischeri) was at least 4.5 times lower than that of surfactins. But they also showed that the EC50 on Daphnia magna for mycosubtilins (8 mg/L) is 3 times lower than that for surfactins (26 mg/L) (Deravel et al. 2014). In another study on iturin A, its acute toxicity was investigated in rats for 28 days. Iturin A showed no toxic effects on the lungs, heart and kidneys after the 28-day treatment, as well as after a recovery period (14 days) (Dey et al., 2016). More recently, a cytotoxicity study was carried out using a mixture of three lipopeptide families (surfactins, fengycins and mycosubtilins) on two different cell lines: Caco-2 and Vero cells. Results showed that the mixture of mycosubtilin isoforms exhibited an IC₅₀ between 10 and 20 mg/L on these two cell lines (Kourmentza et al., 2021).

Today, there is a need for large quantities of antifungal molecules with improved properties to make these biological alternatives economically viable on an industrial scale.

### BRIEF SUMMARY

New Bacillus subtilis strains have been developed that are capable of producing new mycosubtilin isoforms that are both more effective as an antifungal agent and less cytotoxic than mycosubtilins described to date.

The present disclosure relates to two new Bacillus subtilis strains genetically modified by 7 mutations to produce new mycosubtilin isoforms. These two strains are registered with the CNCM under numbers CNCM I-5565 and CNCM I-5679.

The present disclosure relates to 2 new mycosubtilin isoforms, namely Gln1-C16 and Gln1-C17, and compositions containing them.

The present disclosure also relates to the use of new isoforms as antifungal agents, in particular, against B. cinerea, Aspergillus sp and Z. tritici strains.

Finally, the present disclosure relates to a method for producing at least one of these new isoforms by culturing a strain of Bacillus subtilis genetically modified in accordance with the present disclosure.

The present disclosure gives access to surfactants suitable for use in agriculture, namely compounds that are non-toxic and available in large quantities.

Indeed, the new mycosubtilin isoforms presented here exhibit superior antifungal activities to those of the mycosubtilins disclosed to date. In particular, the GlnlC16 isoform exhibits greater antifungal activity than the C16 isoform. Furthermore, these new isoforms exhibit less cytotoxicity than the same known mycosubtilins.

In agriculture, these new isoforms can be used in the production of biopesticides or biosurfactants for the phytosanitary industry for biocontrol of plant diseases or post-harvest treatment, as well as for plant growth stimulation.

The properties of such surfactants make them particularly useful in other fields, such as the food, cosmetics, chemical, medical, pharmaceutical, detergent, oil and environmental industries.

### BRIEF DESCRIPTION OF THE DRAWINGS

FIG. 1 shows a classical formula for mycosubtilin A-C17 (n=13).

FIG. 2 shows a representation of the preparative HPLC chromatogram. Peaks P3, P5, P6, P7 and P8 are classic mycosubtilin isoforms, while peaks P1, P2 and P4 are new mycosubtilin isoforms. The modified amino acid in the peptide ring has been labeled.

FIG. 3 shows the formula for mycosubtilin isoform Gln3-C16.

FIG. 4 shows the formula for the new mycosubtilin isoform Gln1-C16.

FIG. 5 shows the formula for the new mycosubtilin isoform Gln1-C17.

FIG. 6 shows the quantitative analysis of mycosubtilin produced by the new modified Bacillus subtilis strains.

FIG. 7 shows the percentage cell viability test of vero cells treated with different mycosubtilin isoforms at different concentrations of each isoform.

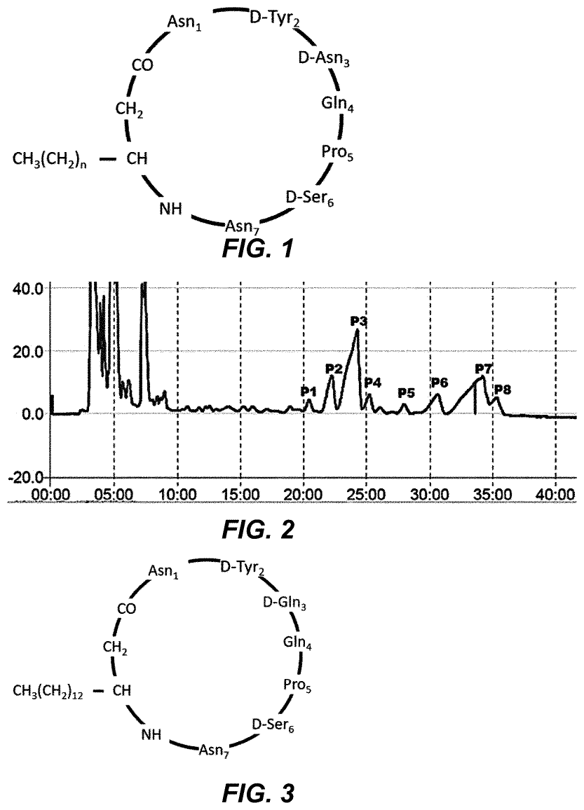

### DETAILED DESCRIPTION

A first object of the present disclosure relates to the Bacillus subtilis strain deposited on Jul. 30, 2020, under number CNCM I-5565 at the Collection nationale de cultures de micro-organismes [National Collection of Microorganism Cultures] (CNCM) of the Institut Pasteur (Paris, France).

A second particular strain according to the present disclosure is the Bacillus subtilis strain deposited on May 5, 2021, under number CNCM I-5679 in the Collection Nationale de Cultures de Microorganismes (CNCM) of the Institut Pasteur (Paris, France).

Strain I-5565 has 7 mutations responsible for a change in metabolism, leading to an increase in mycosubtilin production and the appearance of new isoforms, in particular, Gln1-C16 and Gln1-C17.

Strain I-5679 differs from strain I-5565 in the nature of the promoter enabling mycosubtilin expression: strain I-5565 comprises the native promoter, whereas strain I-5679 has been modified by replacing the endogenous promoter with the constitutive promoter PrepU. The change of promoter led to an increase in the overall production of mycosubtilin: both the conventional form of mycosubtilin and the new isoforms produced by strain I-5565.

The present disclosure also relates to a strain as defined above (strain I-5565) modified by replacement of the endogenous promoter by the constitutive PrepU promoter, wherein it is the Bacillus subtilis strain deposited on May 5, 2021, under number CNCM I-5679 in the Collection Nationale de Cultures de Microorganismes (CNCM) of the Institut Pasteur (Paris, France).

The strains according to the present disclosure are genetically modified Bacillus sp. strains wherein they comprise at least the following mutations:
replacement of a serine by an asparagine due to a mutation at position 1118 of the resE_I gene;
replacement of aspartic acid by asparagine due to a mutation at position 3466 of the rpoC gene;
replacement of an alanine by a valine due to a mutation in position 886 of the addB gene;
replacement of a glutamic acid by a lysine due to a mutation at position 121 of the araA gene;
replacement of a proline by a serine due to a mutation in position 154 of the cotY gene;
replacement of a serine by a phenylalanine due to a mutation in position 32 of the 03427gene; and
replacement of a serine by a phenylalanine due to a mutation in position 92 of the 03457gene.

Mutations in these genes are mainly nucleotide mutations.

An example of each of the mutated genes is shown in the sequence listing:
the mutated resE_I gene is represented by the sequence SEQ ID NO. 1;
the mutated rpoC gene is represented by the sequence SEQ ID NO. 2;
the mutated addB gene is represented by the sequence SEQ ID NO. 3;
the mutated araA gene is represented by the sequence SEQ ID NO. 4;
the mutated cotY gene is represented by the sequence SEQ ID NO. 5;
the mutated 03427 gene is represented by the sequence SEQ ID NO. 6; and
the mutated 03457 gene is represented by the sequence SEQ ID NO. 7.

Other mutations are possible as the genetic code is degenerated. A person skilled in the art will be able to identify alternative mutations to those disclosed in these sequences.

It has been shown that the combination of these 7 mutations leads to an overall increase in mycosubtilin production, and, in particular, to the production of new mycosubtilin isoforms, such as Gln1-C16 and Gln1-C17.

The seven genes modified in the Bacillus sp. strains according to the present disclosure are as follows:
resE is a gene that codes for a protein similar to that found in two-component signal transduction systems, and plays a regulatory role in respiration. ResE is involved in the global regulation of aerobic and anaerobic respiration in B. subtilis. ResE (as well as ResD) are two-component regulatory proteins required for transcriptional activation of Fnr under oxygen limitation in B. subtilis. (Sun et al., 1996). Mycosubtilin production is strongly affected by oxygenation conditions (Guez et al., 2008).
rpoC′ is a gene responsible for the beta subunit of DNA-directed RNA polymerase. A mutation of rpoC leads to elevated expression of stress-sensitive regulators, including those of extracytoplasmic function (ECF) σ factor (σM, σW and σX) and general stress σ factor (σB). SigB (σB) plays an important role in the adaptive response, including the pathogen presence response leading to a positive impact on the antifungal molecules produced by Bacillus species. (Lee et al., 2013; Rodriguez Ayala et al., 2020).
araA is a gene encoding L-arabinose isomerase. This gene is repressed by high glucose concentration (Sá-Nogueira et al., 1997).
addB is a gene that contributes to DNA repair and recombination.

The enzyme is functional as a heterodimer of the AddA and AddB subunits, is a fast, processive DNA helicase, and catalyzes DNA unwinding (Yeeles et al., 2009). A mutation in this gene can have an impact on enzyme activity (Haijema et al., 1996).
cotY is the gene that codes for the spore coat protein Y. This gene is part of the sigh regulator and there is a SigE binding site in the promoter region of the mycosubtilin operon. (Wu et al., 2015).
The 03427 gene encodes a protein whose function is not precisely described in the literature, but which may be related to the Rap protein. Rap proteins in B. subtilis regulate the phosphorylation level or DNA-binding activity of response regulators such as Spo0F, involved in sporulation initiation, or ComA, regulating competence development (Diaz et al., 2012). Rap proteins are also involved in the expression of genes encoding lipopeptide biosynthesis.
The 03457 gene encodes a protein whose function is unknown, but which may be linked to the transport of secondary metabolites.

A second object of the present disclosure relates to a new mycosubtilin isoform chosen from the Gln1-C16 and Gln1-C17 isoforms.

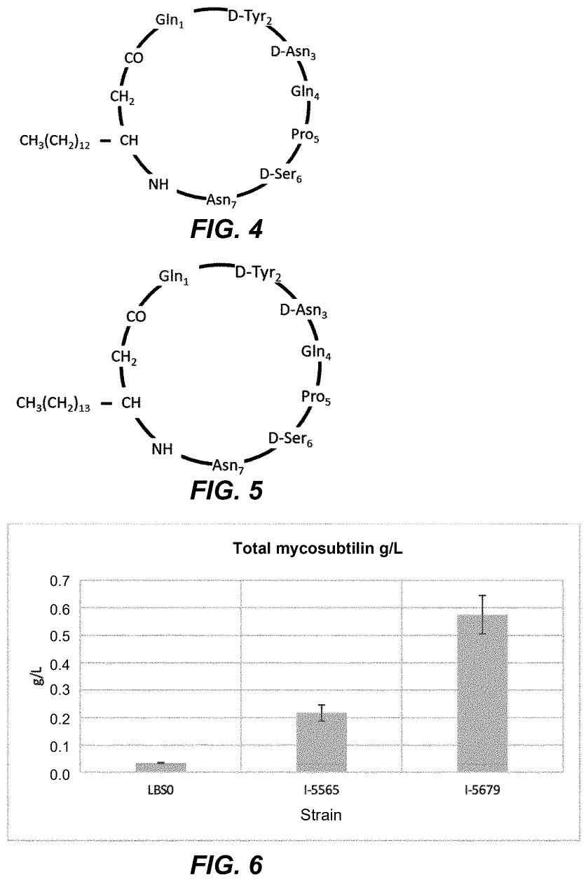

These molecules are shown in FIGS. 4 and 5, respectively.

It should be noted that strains I-5565 and I-5679 produce 13 other new minor mycosubtilins (in terms of production level), namely: Gln7-C16; Gln1, Gln3-C16; Gln3, Gln7-C16; Gln1-C16; Gln1, Gln3, Gln7-C16; Gln7-C17;

### Gln1, Gln3-C17; Gln3, Gln7-C17; Gln1-C17; Gln3-C18; Gln7-C18; Gln1-C18 and A-C19.

A second object of the present disclosure relates to a composition comprising at least one mycosubtilin chosen from the Gln1-C16 and Gln1-C17 isoforms.

A composition according to the present disclosure comprises one, two or three isoforms of mycosubtilin, namely:
the Gln1-C16 isoform;
the Gln1-C17 isoform;
the Gln1-C16 and Gln3-C16 isoforms;
the Gln1-C17 and Gln3-C16 isoforms;
the Gln1-C16 and Gln1-C17 isoforms; and
the Gln1-C16, Gln1-C17 and Gln3-C16 isoforms.

The Gln3-C16 isoform is shown in FIG. 3.

In a preferred embodiment of the present disclosure, the composition comprises at least the Gln1-C16 isoform.

In addition, a composition according to the present disclosure may contain at least one of the novel mycosubtilins selected from Gln7-C16; Gln1, Gln3-C16; Gln3, Gln7-C16; Gln1-C16; Gln1, Gln3, Gln7-C16; Gln7-C17; Gln1, Gln3-C17; Gln3, Gln7-C17; Gln1-C17; Gln3-C18; Gln7-C18; Gln1-C18 and A-C19, as well as any other known mycosubtilin.

Such a composition may also comprise lipopeptides other than mycosubtilin (which is an iturin), including:
molecules in the iturin family, such as iturin A, mojavensin, and bacillomycins A, B, C, D, F and L;
molecules of the surfactin family, such as surfactins A, B or C, lichenysin and pumilacidin;
molecules in the fengycin family, such as fengycins A and B, plipastatins A and B and agrastatins A and B.

Such a composition may also comprise other components such as other surfactant molecules, preservatives and adjuvants.

Surfactant molecules include amphiphilic molecules, surfactants, lipopeptides (e.g., surfactin, fengycin), chemical surfactants and biological surfactants (e.g., rhamnolipids, polysaccharides), etc.

Surfactants include heparin, hyaluronic acid, dextran, amylose, chitosan, anionic surfactants derived from amino acids, non-ionic surfactants derived from polyglycosides, hydrotropic surfactants, lipopeptides such as surfactin isomers and/or fengycin (or plipastatin) isomers, rhamnolipids and vegetable oils.

Surfactants from the non-ionic surfactant family are chosen, for example, from: fatty alcohol axalkylate, pentylene glycol and its derivatives, hydrotropes of the alkylpolyglycoside type (alkypolyglycoside and alkylethoxypolyglycoside), polyglycoside-type texture agent molecules (xanthan gum, gum arabic, tragacanth gum, guar gum, locust bean gum, tamarind gum, pectin, gellan gum, carrageenates, agar-agar, alginates).

Surfactants from the anionic surfactant family are chosen, for example, from surfactins, fengycins, sodium laureth sulfates and its derivatives, or amino acid derivatives. The surfactant may also be a sulfonate or its derivative.

In another embodiment, the surfactant is a lauryl ether sulfate with the formula (I):

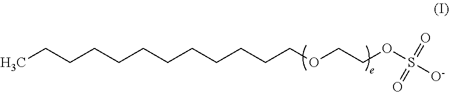

wherein o is an integer between 1 and 5; or an agriculturally acceptable salt, metal complex or metalloid complex thereof. The surfactant may also be a fatty alcohol ether sulfate with a C12-C14 fatty alcohol, or a mixture thereof.

In one aspect, the surfactant is a sulfonate of formula (II):

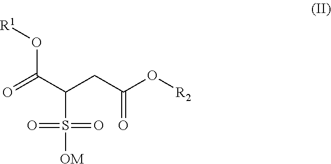

wherein R1 and R2 are independently a linear or branched C1-20 alkyl or a linear or branched C2-20 alkene; and M is H+, Li+, Na+, K+ or (C1-8 alkyl)4N+.

In some embodiments, R1 and R2 are independently linear or branched C8 alkyl. In other embodiments, the sulfonate is dioctyl sulfosuccinate; 1,4-bis(2-ethylhexoxy)-1,4-dioxobutane-2-sulfonate; or a Li+, Na+, K+ or (C1_8 alkyl)4N+ salt thereof, chosen, for example, from modified (acidified, methylated, esterified) oils and oil extracts, in particular, from: almond, peanut, argan, avocado, rapeseed, ricin, lorenzo, neem, hazelnut, cashew nut, macadamia nut, olive, pistachio, rice, oleic sunflower, camelina, linseed, borage, safflower, hemp, cotton, wheat germ, corn, walnut, poppy seed, evening primrose, barley, pumpkin seed, grapeseed, pea, sesame, soybean, sunflower.

Among adjuvants, some can facilitate the penetration of the composition into the plant (such as oils), others increase the contact surface between the plant leaf and the composition (wetting agents), others can absorb moisture from the air and thus combat desiccation (salts), still others can fix the composition to the leaves so as to limit leaching and volatilization. Adjuvants therefore need to be adapted to the mode of action of the compositions (root, contact, systemic or penetrating), the type of product formulation and the type of plant targeted (hairy or hairless leaves, cuticle thickness, plant stage, stomatal position, etc.).

In a preferred embodiment the composition of the present disclosure comprises:
(i) at least one mycosubtilin chosen from the Gln1-C16 and Gln1-C17 isoforms, preferably at a concentration of at least 0.5% (w/w), preferably from about 0.5% (w/w) to about 10% (w/w), more preferably from about 1% (w/w) to about 5% (w/w), most preferably about 2.5% (w/w); and
(ii) at least one additional surfactant, preferably at a concentration of at least 1% (w/w), preferably from about 1% (w/w) to about 20% (w/w), more preferably from about 5% (w/w) to about 15% (w/w), most preferably about 10% (w/w).

In preferred embodiment at least one additional surfactant is a fatty alcohol ether sulfate, preferably lauryl ether sulfate, or a sulfonate, preferably dioctyl sulfosuccinate. In another preferred embodiment the composition comprises a fatty alcohol ether sulfate, preferably lauryl ether, sulfate and a sulfonate, preferably dioctyl sulfosuccinate.

A third object of the present disclosure relates to the use of at least one mycosubtilin selected from the Gln1-C16 and Gln1-C17 isoforms or of a composition comprising such a mycosubtilin (as previously disclosed) as an antifungal agent.

Both isoforms are effective against an ascomycete fungus, in particular, B. Cinera, Aspergillus sp. and Z. tritici.

In a particular embodiment, this use consists in using at least one mycosubtilin corresponding to the Gln1-C16 isoform as an antifungal agent against the B. Cinera strain.

In another particular embodiment, this use consists in using at least one mycosubtilin corresponding to the Gln1-C17 isoform as an anti-fungal agent against the Aspergillus sp. strain.

In yet another particular embodiment, this use consists in using at least one mycosubtilin corresponding to one of the Gln1-C16 or Gln3-C16 isoforms as an antifungal agent against the Z. tritici strain. One particular embodiment consists in using Gln1-C16 or Gln1-C17 in combination with Gln3-C16 as an antifungal agent against the Z. tritici strain.

A fourth object of the present disclosure relates to a method for producing an antifungal composition consisting in cultivating a genetically modified Bacillus strain as defined above.

In a particular embodiment, the production method produces at least one new mycosubtilin isoform selected from Gln1-C16 and Gln1-C17 isoforms by cultivating a genetically modified Bacillus strain as defined above.

Sequentially, this method consists in:
culturing a Bacillus sp. strain according to the present disclosure;
incubating this strain in a suitable culture medium; and
harvesting the strain and/or culture supernatant.

This method may continue with one or more of the following steps:
purifying the mycosubtilins (globally or differentiated by isoform);
concentrating the solution containing the mycosubtilin or the isoforms of interest of the mycosubtilin; and
dehydrating the solution containing the mycosubtilin or the isoforms of interest of the mycosubtilin.

The new mycosubtilin isoforms disclosed above and the compositions containing them can be used in many applications because of their surfactant and/antifungal properties and their absence or low level of toxicity:
in agriculture: production of biopesticides or biosurfactants for the phytosanitary industry for biocontrol of plant diseases or post-harvest treatment, or as a plant growth stimulation agent;
in the food industry: preservative;
in cosmetics: preservative and formulation agent;
in chemistry;
in the medical and pharmaceutical fields: antifungal action or prevention, preservative;
as a detergent;
in the oil industry;
in the field of environmental protection: biological pest control, non-toxic formulation agent.

They can therefore be used in non-therapeutic applications such as agriculture and the food, chemical, detergent, oil and environmental industries, or in medical applications such as cosmetics, medicine and pharmaceuticals.

The present disclosure will be better understood upon reading the examples that follow, provided by way of illustration, and in no way considered to be limiting on the scope of the present disclosure.

### EXAMPLES

### Example 1: Construction of Bacillus subtilis Strains I-5565 and I-5679

Bacillus subtilis strain I-5679 is derived from strain I-5565; the constructions of these strains are described below.

A first strain deposit was made on Jul. 30, 2020, under number CNCM I-5565 at the Collection Nationale de Cultures de Microorganismes (CNCM) of the Institut Pasteur (Paris, France). A second strain deposit was made on May 5, 2021, under number CNCM I-5679 in the Collection Nationale de Cultures de Microorganismes (CNCM) of the Institut Pasteur (Paris, France).

1.1 Procedure for Obtaining B. subtilis Strain I-5565

Strain I-5565 was constructed from a B. subtilis strain deposited on Feb. 6, 2020, under CNCM number I-5487 at the Collection Nationale de Cultures de Microorganismes (CNCM) of the Institut Pasteur (Paris, France).

In this strain I-5487, seven genes were modified (resk, rpoC, addB, araA, cotY, 03427 and 03457) by means of a point modification, that is to say a change in a single nucleotide. The deletion or modification of a single nucleotide (point mutation) can change the amino acid sequence, resulting in a new phenotype in the mutant strain. Various genetic engineering techniques can be used to achieve this type of genetic mutation:
The CRISPR-Cas9-mediated genome engineering method surpassed previous methods in terms of accuracy and enabled the creation of reliable mutants without foreign DNA in many wild-type bacterial strains, including B. subtilis, with point mutation efficiencies of up to ˜68%. This method can create a new phenotype without introducing any foreign DNA into the genome (So et al., 2017).
Site-directed mutagenesis is one of the foundations of modern molecular biology, enabling perfect control of protein sequences. Several strategies have been developed, with the QUIKCHANGE™ site-directed mutagenesis system developed by Stratagene (La Jolla, CA) probably being the most widely used. QUIKCHANGE™ works by using a complementary primer pair with a mutation. In a series of PCR cycles, these primers hybridize to the template DNA, replicating the plasmid DNA with the mutation. The mutant DNA product exhibits a strand break (nick). The resulting DNA pool (mutant and parental) is then treated with Dpnl to destroy methylated parental DNA from newly synthesized unmethylated mutant DNA and transformed into E. coli cells where the break is ligated by host repair enzymes. (Liu & Naismith, 2008).
Targeted mutagenesis using a mutagenic agent. This method relies on the use of chemical mutagenic agents (ethyl methane sulfonate (EMS) and methyl methane sulfonate (MMS)) or physical agents such as strong exposure to ultraviolet (UV) light. genetic modification occurs, creating a change in phenotype. EMS will be chosen to target G-C rich regions, while UV will be chosen to target an A-T rich region.

After genetic modification, using the mutation procedure disclosed above, interesting mutants were obtained. The method used for mutant strain screening comprises diluting one unit volume of mutant library stock in one unit volume of physiological water. The cell suspension is spread on nutrient agar plates (provided by Condalab) to obtain single colonies. Several single colonies are then picked using the Qpix 460 (from Molecular Devices) and inoculated into nutrient culture medium. After incubation at 30° C. for 24 h, the cells are harvested, and the supernatant is used for mycosubtilin analysis by reverse-phase high-performance liquid chromatography as disclosed in Example 2. In a different embodiment, other culture media can be used, such as LB medium, Landy medium or Cooper medium.

Using this methodology, after several rounds of point mutation procedures, B. subtilis strain I-5565 was selected. Genome sequencing using Illumina Paired end 2*150 bp (Library prepared fowling Nextera XT) of this strain showed changes in the sequence of these seven genes as disclosed below.

1.2 Genetic Characterization of B. subtilis Strain I-5565

The genome of B. subtilis strain I-5565 was sequenced using Illumina Paired end 2*150 bp (library prepared fowling Nextera XT). Genome sequencing results confirm changes in the nucleotide sequence of seven genes: resE, rpoC, addB, araA, cotY, 03427 and 03457.

The impact of these nucleotide changes was then analyzed and confirmed on the primary structure of the corresponding proteins, and is disclosed below:
change in amino acid sequence from serine to asparagine due to a mutation in the RseE_1 gene at position 1118.
change in amino acid sequence by replacement of the aspartic acid by an asparagine in the RpoC gene at position 3466.
change in amino acid sequence from alanine to valine due to a mutation in the AddB gene at position 886.
change in amino acid sequence by replacement of a glutamic acid by a lysine due to a mutation in the AraA gene at position 121.
change in amino acid sequence by replacement of a proline by a serine due to a mutation in the CotY gene at position 154.
change in amino acid sequence by replacement of a serine by a phenylalanine due to a mutation in gene 03427 at position 32.
change in amino acid sequence by replacement of a serine by a phenylalanine due to a mutation in gene 03457 at position 92.

Compared with B. subtilis LBS0, Bacillus subtilis strain I-5565 produces more mycosubtilin, as shown in Example 2, and various new isoforms.

1.3 pLIP1 Plasmid Construction Protocol

To replace the native mycosubtilin operon promoter in B. subtilis strain I-5565, a hybrid plasmid containing &pbp-P_{repU}-neo-EfenF and rep (R6K) was first constructed as follows. The bLIP1 plasmid is digested with the restriction enzymes Xmil (Thermo Scientific; reference FD1484) and Pael (Thermo Scientific; reference FD0604) to extract and purify a 4.5 kilobase-pair (Kb) fragment following the protocol of the GeneJET gel extraction kit (Thermo Scientific; reference K0692). The pbp gene fragment (950 base pairs) is amplified from B. subtilis I-5565 using primers containing similar restriction sites. The amplified fragment is extracted and purified. Ligation of the 4.5 kb fragment and the 950 bp PCR fragment is performed following the protocol of the Rapid DNA Ligation Kit (Thermo Scientific; reference K1422). The ligation product is transformed into ready-to-use competent E. coli JM 109 cells (Promega, reference L2005). Clones are selected on Luria-Bertani medium containing agar and 50 μg/ml neomycin/kanamycin. The final plasmid from positive clones is extracted following the protocol of the GeneJET plasmid mini-preparation kit (Thermo Scientific; reference K0503). The final plasmid obtained is called pLIP1 (5.5 kb).

1.4 Protocol for Obtaining B. subtilis I-5679

The B. subtilis I-5565 strain is then transformed with the pLIP1 plasmid using the natural transformation protocol. Clones are selected on Luria-Bertani medium containing agar and 20 μg/ml neomycin/kanamycin. The positive clone is verified by colony PCR. The final construction is known as B. subtilis I-5679. Compared with B. subtilis I-5565, Bacillus subtilis I-5679 strain produces a higher concentration of mycosubtilin and a greater number of the various new isoforms presented in Example 2.

### Example 2: Production and Purification of Mycosubtilin from Newly Modified B. Subtilis Strains

### 2.1 Composition of the Modified Landy Medium

The composition of the modified Landy medium is as follows: glucose, 60 g/l; ammonium sulfate, 8 g/l; yeast extract, 4 g/l; MgSO₄, 0.25 g/l; K₂HPO₄, 1 g/l; KCl, 0.5 g/l; CuSO₄·5H₂O, 1.6 mg/l; FeSO₄·7H₂O, 1.2 mg/l; MnSO₄·H₂O, 0.4 mg/l.

### 2.2 Stock Solutions

To ensure reproducibility of the medium composition, sterile concentrated solutions are produced. A 10× glucose solution (400 g/l) is sterilized by autoclaving at 121° C. for 10 minutes. A 10× ammonium sulfate solution (80 g/l); a 10× yeast extract solution (40 g/l); a 10× mineral salt solution No. 1 (KHPO 10 g/l; MgSO 2.5 g/l; KCl, 5 g/l); a 1000× mineral salt solution No. 2 (CuSO₄·5H₂O, 1.6 g/l; FeSO₄·7H₂O, 1.2 g/l; MnSO₄·H₂O, 0.4 g/l) was acidified with concentrated sulfuric acid for total dissolution of the salts, and all these stock solutions were sterilized by autoclaving at 121° C. for 20 minutes.

2.3 Production of One Liter of Landy Medium Modified with MOPS

A 150 ml glucose solution is taken under sterile conditions and poured into a 1 L reagent bottle. 100 ml yeast extract solution, 100 ml ammonium sulfate solution, 100 ml mineral solution No. 1, and finally 1 ml mineral solution No. 2 are added successively to each solution under sterile conditions. A 20×MOPS (2M) buffer is produced by dissolving 3-N-Morpholinopropanesulfonic acid (MOPS) (Sigma, reference M3183) in water, pH adjusted to 7.0 by NaOH. The solution is then sterilized on a 0.2 μm porosity filter. To produce 1 liter of modified Landy medium buffered to 100 mM with MOPS, 50 ml of 20×MOPS was added to the mixture.

The final pH was adjusted to 7.0 using a sterile 6M NaOH solution. The volume is brought up to 1 liter with sterile water.

### 2.4 Preparation of an Inoculum

The inoculum was prepared from a stock of strains stored at −80° C. in 25% glycerol. Glycerol stock was inoculated into Luria-Bertani (LB) broth for 7-8 hours. The culture was then spread on Luria-Bertani (LB) agar plates containing 20 μg/ml kanamycin (kanamycin sulfate, Sigma, reference 60615) to obtain a single colony. The plate is incubated at 37° C. overnight. A P1 preculture is then produced with a single colony from the plate grown overnight inoculated into a final volume of 10 ml Luria-Bertani (LB) broth containing 20 μg/ml kanamycin contained in a 100 ml Erlenmeyer flask. The culture is incubated at 30° C. with 200 rpm agitation for 10 to 14 hours. After the P1 incubation period of the preculture, the culture is centrifuged at 2000 g for 10 minutes at 25° C. The cells are washed with physiological water and finally suspended in sterile physiological water. The suspension is now ready for inoculation. A P2 preculture is produced with an inoculation OD₆₀₀ of 0.2 in a final volume of 100 ml of modified Landy medium contained in a 1 L Erlenmeyer flask. The culture is incubated at 30° C. with 200 rpm agitation for 7 to 8 hours.

### 2.5 Cultures in Erlenmeyer Flasks

Prior to inoculation, P2 preculture cells are harvested and washed with physiological water, then suspended in sterile physiological water. This suspension is used for inoculation. The inoculation OD₆₀₀ is between 0.1 and 0.2. The volume of the Erlenmeyer flasks is 1 L containing 100 ml of modified Landy medium. Similarly, ten 1 L Erlenmeyer flasks containing 100 ml of modified Landy medium are prepared for a high concentration of mycosubtilin. The culture is incubated at 30° C. with 200 rpm agitation for a maximum of 72 hours. After the incubation period, the culture supernatant is used for lipopeptide analysis.

### 2.6 Concentration and Purification of Mycosubtilin

Cultures are mixed with acetonitrile (VWR Chemical, order No. 83639.320) to a final concentration of 50% acetonitrile. The mixture is centrifuged at 8500 g for 10 minutes at 25° C. The cells are separated and the supernatant is used to concentrate mycosubtilin using a laboratory rotary evaporator (Buchi, Rotavapor R300). The sample was concentrated to a final concentration of 3 g/L.

### 2.7 Quantitative Analysis of Mycosubtilin by UPLC

Samples were analyzed using a complete Waters UPLC system (Aqcuity H class plus) (Waters SAS, Guyancourt, France) using a C18 column (2.1×150 mm, CORTECS. UPLC. C18.1.6 μm).

For mycosubtilin analysis: 3 μL of purified sample were injected and compared to a 500 mg/Liturin A standard (11774, Sigma-Aldrich, St. Louis, MO, U.S.A.) at a flow rate of 0.4 mL/min. Elution was carried out in isocratic mode using a water/acetonitrile/formic acid 60/40/0.1 (v/v/v) solvent. The retention time and second derivative of the spectrum between 200 and 400 nm for each peak were automatically analyzed using Empower 3 software to identify the eluted molecules.

As shown in FIG. 6, cultures of each strain were analyzed in triplicate and the mycosubtilin production of the three strains (LBS0, I-5565 and I-5679) was compared.

As shown in FIG. 6, mycosubtilin production increased 6-fold in strain I-5565 compared with its parent strain LBS0, while mycosubtilin production in strain I-5679 (where the native mycosubtilin promoter has been replaced by the constitutive prepU promoter in I-5565) was 28.5 and 2.5 times higher than in LBS0 and I-5565, respectively. Comparing these results with those obtained with the B. subtilis BBG125 strain previously obtained by suppressing surfactin production and inserting the PrepU promoter in front of the mycosubtilin gene (WO2013/050700), strain I-5565 produces 5 times more than strain BBG125 and strain I-5679 produces 12 times more than strain BBG125. In conclusion, interruption of the resE, rpoC, addB, araA, cotY, 03427 and 03457 genes in strain I-5565 unexpectedly led to an increase in production well in excess of previous results. More importantly, the combination of these seven gene interruptions and the PrepU promoter insertion in strain I-5679 further increased mycosubtilin production by a factor of 2.5 compared with strain I-5565.

### 2.8 Preparative HPLC Analysis of Mycosubtilin

The PuriFlash-interchim preparative HPLC column is used to separate isoforms.

Purification was carried out on the US5C18HQ-250/300 interchim column (UPTISPHERE STRATEGY C18-HQ 5 μm 250×30 mm HPLC COLUMN).

The parameters used for preparative HPLC are as follows:
Injection volume: 10 ml;
Mobile phase: water/acetonitrile with 0.1 TFA;
Flow rate: 35 ml/min;
Fraction volume: 14 ml and threshold was 3 mAU; and
Detector: 214 nm.

The program used for isoform separation is shown in Table 1 below:

TABLE 1
Size of acetonitrile used for the separation
of mycosubtilin isoforms by preparative HPLC.

| Time (minutes) | Acenitrile (%) | Water (%) |
|---|---|---|
| 0 | 20 | 80 |
| 2 | 20 | 80 |
| 3 | 43 | 57 |
| 43 | 43 | 57 |
| 44 | 90 | 10 |
| 46 | 90 | 10 |
| 47 | 20 | 80 |
| 49 | 20 | 80 |

The different peaks representing the different mycosubtilin isoforms are shown in FIG. 2. Each peak was collected in several 14 ml tubes. The number of tubes depends on the volume of the peak. A total of 5 runs were carried out (10 ml of sample for each run). Each isoform sample was then analyzed by mass spectrometry (Q-tof analysis) as disclosed in Example 3. Antifungal analysis was carried out on the different isoforms as disclosed in Example 4. Cytotoxicity analysis was also carried out on the different isoforms as disclosed in Example 5.

### Example 3: Qualitative Analysis of Mycosubtilin Produced by Newly Modified B. Subtilis Strains

The parameters used for LC/MS-Qtof analysis are as follows:

UPLC experiments were carried out on Agilent 1290 Infinity II 2D-LC technologies. A C18 Acquity UPLC BEH column (2.1×50 mm×1.7 μm; Waters) was used at a flow rate of 0.3 ml/min and a temperature of 40° C. The injection volume was 20 μl and the diode array detector (DAD) scanned a wavelength spectrum between 190 and 600 nm. Agilent OpenLab CDS ChemStation software and Agilent 1290 Infinity 2D-LC acquisition software were used for LC analysis.

The parameters used for UPLC are as follows:
Injection volume: 10 μl;
Column temperature: 40° C.; and
Mobile phase: Water/Acetonitrile with 0.1% formic acid.

The program used for isoform separation is shown in Table 2 below:

TABLE 2
Acetonitrile content used for LC/MS-Qtof
analysis of mycosubtilin isoforms

| Time (minutes) | Acenitrile (%) | Water (%) |
|---|---|---|
| 0 | 37 | 63 |
| 5 | 37 | 63 |
| 7.5 | 45 | 55 |
| 8 | 100 | 0 |
| 10 | 100 | 0 |
| 10.5 | 37 | 63 |

LC/MS-Qtof analyses were performed using the 1290 Infinity II coupled to the Jet Stream ESI-Q-TOF 6530 (Agilent Technologies) in negative mode using the following parameters: capillary voltage: 3.5 kV; nebulizer pressure: 35 psig; drying gas: 8 L/min; drying gas temperature: 300° C.; sheath gas flow rate: 11 L/min; sheath gas temperature: 350° C.; fragmenter voltage: 175 V; skimmer voltage: 65 V; ctopole RF: 750 V. Accurate mass spectra were recorded in the range m/z=100-1200. In short, 10 μl of sample was injected and separated using a C18 Acquity UPLC BEH (2.1×50 mm×1.7 μm; Waters) using 0.1% formic acid (FA) as solvent A and ACN+0.1% FA as solvent B. The gradient started with 0% solvent B and increased to 30% within 5 min. Data were processed using MassHunter Qualitative Analysis software (Agilent Technologies). Table 3 shows the new mycosubtilin C16 and C17 isoforms in comparison with the conventional C16 and C17 obtained from LC/MS-Qtof analysis as disclosed above.

TABLE 3
LC/MS-Qtof analysis of mycosubtilin isoforms in various fractions
purified by preparative HPLC (new isoforms in bold)

| Peak | | Fatty | | | | | | | | |
|---|---|---|---|---|---|---|---|---|---|---|
| number | Variants | acid | AA1 | AA2 | AA3 | AA4 | AA5 | AA6 | AA7 | m/z |
| 1 | Gln3-C16 | C16 | Asn | Tyr | Gln | Gln | Pro | Ser | Asn | 1085 |
| 2 | Gln1-C16 | C16 | Gln | Tyr | Asn | Gln | Pro | Ser | Asn | 1085 |
| 3 | A-C16 | C16 | Asn | Tyr | Asn | Gln | Pro | Ser | Asn | 1071 |
| 4 | Gln1-C17 | C17 | Gln | Tyr | Asn | Gln | Pro | Ser | Asn | 1099 |
| 5 | A-C17 | C17 | Asn | Tyr | Gln | Gln | Pro | Ser | Asn | 1085 |
| 6 | A-C17 | C17 | Asn | Tyr | Asn | Gln | Pro | Ser | Asn | 1085 |
| 7 | A-C17 | C17 | Asn | Tyr | Asn | Gln | Pro | Ser | Asn | 1085 |
| 8 | A-C18 | C18 | Asn | Tyr | Asn | Gln | Pro | Ser | Asn | 1099 |

In addition to the conventional C16 isoform of mycosubtilin (C16-myco), the C17 isoform of mycosubtilin (C17-myco) and the C18 isoform of mycosubtilin (peak 3, peak 5, peak 6, peak 7 and peak 8), three new isoforms were detected. Peaks 1, 2 and 4, corresponding to C16-Gln1 and C17-Gln1, are new mycosubtilin isoforms. Asparagine is replaced by Glutamine in third position for peak 1, while Asparagine is replaced by Glutamine in third position for peaks 2 and 4.

TABLE 4
LC/MS-Qtof analysis of mycosubtilin isoforms on the complete supernatant of
B. subtilis strains I-5565 and I-5679 (new isoforms in bold) (AA = amino acid).

| | Fatty | | | | | | | | |
|---|---|---|---|---|---|---|---|---|---|
| Variants | acid | AA1 | AA2 | AA3 | AA4 | AA5 | AA6 | AA | m/z |
| Gln3-C16 | C16 | Asn | Tyr | Gln | Gln | Pro | Ser | Asn | 1085 |
| Gln7-C16 | C16 | Asn | Tyr | Asn | Gln | Pro | Ser | Gln | 1085 |
| Glnl, Gln3-C16 | C16 | Gln | Tyr | Gln | Gln | Pro | Ser | Asn | 1099 |
| Gln3, Gln7-C16 | C16 | Asn | Tyr | Gln | Gln | Pro | Ser | Gln | 1099 |
| Gln1-C16 | C16 | Gln | Tyr | Asn | Gln | Pro | Ser | Asn | 1085 |
| A-C16 | C16 | Asn | Tyr | Asn | Gln | Pro | Ser | Asn | 1071 |
| Glnl, Gln3, Gln7- | C16 | Gln | Tyr | Gln | Gln | Pro | Ser | Gln | 1113 |
| C16 | | | | | | | | | |
| Gln3-C17 | C17 | Asn | Tyr | Gln | Gln | Pro | Ser | Asn | 1099 |
| Gln7-C17 | C17 | Asn | Tyr | Asn | Gln | Pro | Ser | Gln | 1099 |
| Glnl, Gln3-C17 | C17 | Gln | Tyr | Gln | Gln | Pro | Ser | Asn | 1113 |
| Gln3, Gln7-C17 | C17 | Asn | Tyr | Gln | Gln | Pro | Ser | Gln | 1113 |
| Gln1-C17 | C17 | Gln | Tyr | Asn | Gln | Pro | Ser | Asn | 1099 |
| A-C17 | C17 | Asn | Tyr | Asn | Gln | Pro | Ser | Asn | 1085 |
| A-C17 | C17 | Asn | Tyr | Asn | Gln | Pro | Ser | Asn | 1085 |
| A-C17 | C17 | Asn | Tyr | Asn | Gln | Pro | Ser | Asn | 1085 |
| A-C18 | C18 | Asn | Tyr | Asn | Gln | Pro | Ser | Asn | 1099 |
| Gln3-C18 | C18 | Asn | Tyr | Gln | Gln | Pro | Ser | Asn | 1113 |
| Gln7-C18 | C18 | Asn | Tyr | Asn | Gln | Pro | Ser | Gln | 1113 |
| Gln1-C18 | C18 | Gln | Tyr | Asn | Gln | Pro | Ser | Asn | 1113 |
| A-C19 | C19 | Asn | Tyr | Asn | Gln | Pro | Ser | Asn | 1113 |

In addition to the eight major isoforms purified by preparative HPLC (shown in Table 3), several other minor isoforms were separated by UPLC from the supernatants of strains I-5565 and I-5679. These minor isoforms were then identified and characterized using the highly sensitive MS-Qtof method disclosed hereinbefore. As shown in Table 4, a total of 20 isoforms were identified in the supernatants of the new B. subtilis strains. Among these, 13 new minor isoforms have been discovered, listed here: Gln7-C16; Gln1, Gln3-C16; Gln3, Gln7-C16; Gln1-C16; Gln1, Gln3, Gln7-C16; Gln7-C17; Gln1, Gln3-C17; Gln3, Gln7-C17; Gln1-C17; Gln3-C18; Gln7-C18; Gln1-C18 and A-C19. In addition to all the new isoforms discovered by this work, it was also found that in the overproducing mutants, the adenylation domains of mycosubtilin synthetase can activate both Gln and Asn at positions 1, 3 and 7 of the peptide cycle. The new isoforms are used for activity and cytotoxicity experiments to determine their efficacy. The isoforms are dissolved in DMSO and used for activity studies. The results are disclosed in Examples 4 and 5.

### Example 4: Antifungal Activity of Different Mycosubtilin Isoforms

Three different fungal strains were tested for their antifungal activity: B. cinerea, Aspergillus sp. and Z. tritici. In addition to the two conventional isoforms, three new isoforms are being tested for their antifungal properties. A range of concentrations (μg a.i./ml or ppm) 0; 0.0037; 0.0146; 0.0586; 0.2344; 0.9375; 3.75 and 15 is used to determine the activity coefficient. Based on this concentration range, the EC₅₀ (concentration that reduces fungal strain growth by 50%) and EC95 (concentration that reduces pathogen growth by 95%) were calculated and presented as disclosed in Table 5.

4.1 Antifungal Activity on B. cinerea

The EC₅₀ value for A-C16 is 0.933 ppm, while Gln1-C16 is 1.048 ppm, indicating a 10% lower concentration of the former required to have the same activity as the latter. The EC₉₅ value for A-C16 is 1.457 ppm, while that for Gln1-C16 is 1.282 ppm, indicating a 17% higher concentration of the former required to have the same activity as the latter. This new isoform (Gln1-C16) is effective against this fungal strain.

The other two isoforms tested, Gln3-C16 and Gln1-C17, proved effective against this fungal strain, but at higher concentrations.

### 4.2 Antifungal Activity on Aspergillus sp.

The EC₅₀ value for A-C17 is 1.191 ppm, while that for Gln1-C17 is 1.019 ppm, indicating a 14% higher concentration of the former required to have the same activity as the latter. The EC₉₅ value for A-C17 is 1.905 ppm, while that for Gln1-C17 is 1.290 ppm, indicating a 32% higher concentration of the former required to have the same activity as the latter. This new isoform proves effective against this fungal strain.

The other two isoforms tested, Gln1-C16 and Gln3-C16, proved effective against this fungal strain, but at higher concentrations.

### 4.3 Antifungal Activity on Z. Tritici

The EC₅₀ value for A-C16 is 4.258 ppm, while Gln1-C16 is 1.906 ppm, indicating a 55% higher concentration of the former required to have the same activity as the latter. The EC₉₅ value for A-C16 is 26.106 ppm, while that for Gln3-C16 is 17.874 ppm, indicating a 32% higher concentration of the former to have the same activity as the latter. These isoforms proved effective against this fungal strain.

The other new Gln1-C17 isoform is effective against this fungal strain, but at a higher concentration.

TABLE 5
Antifungal activity of 6 different mycosubtilin
isoforms on three phytopathogenic fungi.

| | Gln3-C16 | Gln1-C16 | A-C16 | Gln3-C17 | Gln1-C17 | A-C17 |
|---|---|---|---|---|---|---|
| EC_{S0} (μg/ml) | | | | | | |
| B. cinerea | 9.918 | 1.048 | 0.933 | 1.646 | 0.920 | 0.467 |
| Aspergillus sp. | 13.148 | 1.691 | 1.062 | 6.335 | 1.019 | 1.191 |
| Z. tritici | 10.077 | 1.906 | 4.258 | 4.314 | 2.156 | 0.943 |
| EC₉₅ (μg/ml) | | | | | | |
| B. cinerea | 14.232 | 1.282 | 1.457 | 3.437 | 1.117 | 0.677 |
| Aspergillus sp. | 14.772 | 3.040 | 1.374 | 13.659 | 1.290 | 1.905 |
| Z. tritici | 17.874 | 24.286 | 26.106 | 16.078 | 17.040 | 18.010 |

### Example 5: Cytotoxicity Tests

The cytotoxicity test is performed using Vero cells and the CyQUANT™ XTT Cell Viability assay kit (supplied by Invitrogen™).

Samples are dissolved in 100% DMSO at a concentration of 20-50 g/L. The final concentration in DMEM medium is 40 μg/L. Cells were prepared in flasks containing DMEM cell culture medium with 10% FBS as follows:

A 175 ml flask containing 20-30 million cells with 25 ml medium and 2 ml trypsin was added. Then the medium was discarded as the cells were attached to the flask wall. Rinse twice with PBS, then trypsin was added to detach cells from the flask wall. The mixture was incubated for 5 min at 37° C. To inactivate trypsin, 6 ml of trypsin was added to the medium (twice the volume of the original trypsin in the medium).

In a 50 ml flask, the medium was centrifuged for 5 min at 300 g. Centrifuge the medium for 5 min at 300 g. The medium was discarded, and the cells were resuspended. 100 μL of cells were added to 100 μL of dye, then 12 μL was used for counting. The percentage of viability was compared with a control of cells without any treatment.

The test is performed in a 96-well plate. A range of concentrations (mg/l) 0.14; 0.28; 0.56; 1.16; 2.25; 4.5 is used to determine the toxicity limit of each isoform.

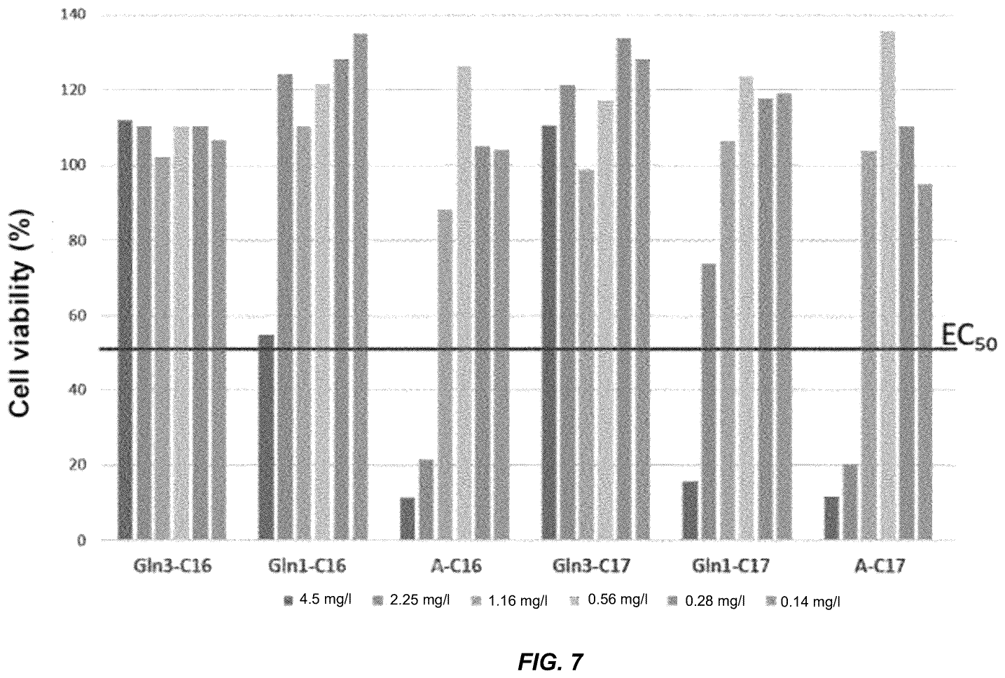

The results presented in FIG. 7 show that the percentage of viable Vero cells is significantly higher in the presence of the new C16 and C17 isoforms compared with the usual A-C16 and A-C17 isoforms. From these data, the EC50 can be determined, the EC50 of Gln3-C16- is over 4.5 mg/L, the EC50 of Gln1-C16 is 4.5 mg/L, the EC50 of A-C16 is 1.7 mg/L, which is at least 2.64 times less than the new C16 isoforms of mycosubtilin. The cytotoxicity profile of C16 isoforms is Gln3-C16<Gln1-C16<A-C16. The C16 isoforms, namely Gln3-C16 and Gln1-C16, are significantly less toxic than the conventional A-C16 isoform. The same profile is observed for mycosubtilin-based C17 isoforms. The EC50 of Gln3-C17 is over 4.5 mg/L, the EC50 of Gln1-C17 is around 3.4 mg/L, the EC50 of A-C17 is around 1.7 mg/L, which is at least 2 times less than the new C17 isoforms of mycosubtilin. For C17 isoforms, the toxicity profile is Gln3-C17<Gln1-C17<A-C17.

What is claimed is:

1. A genetically modified Bacillus sp strain, which is the Bacillus subtilis strain deposited on Jul. 30, 2020, under number CNCM I-5565 at the Collection nationale de cultures de micro-organismes (CNCM) of the Institut Pasteur (Paris, France).

2. The genetically modified Bacillus sp strain of claim 1, modified by replacement of the endogenous promoter by the constitutive PrepU promoter and wherein it is the Bacillus subtilis strain deposited on May 5, 2021, under number CNCM I-5679 in the Collection Nationale de Cultures de Microorganismes (CNCM) of the Institut Pasteur (Paris, France).

3. A mycosubtilin isoform selected from the group consisting of Gln1-C16 and Gln1-C17 isoforms.

4. A composition comprising the mycosubtilin of claim 3.

5. A method of using the at least one mycosubtilin of claim 3 as an antifungal agent, the method comprising applying the mycosubtilin to a surface.

6. A method of killing or inhibiting a fungus, the method comprising contacting the fungus with the mycosubtilin isoform of claim 3.

7. The method of claim 5, wherein at least one mycosubtilin corresponding to the Gln1-C16 isoform is used as an antifungal agent against the B. Cinera strain.

8. The method of claim 5, wherein at least one mycosubtilin corresponding to the Gln1-C17 isoform is used as an anti-fungal agent against the Aspergillus sp. strain.

9. The method of claim 5, wherein at least one mycosubtilin corresponding to the Gln1-C16 isoform is used as an antifungal agent against the Z. tritici strain.

10. A method of killing or inhibiting a Z. tritici strain, the method comprising: contacting the Z. tritici strain with at least one mycosubtilin selected from the group consisting of Gln1-C16 and Gln1-C17 isoforms and the Gln3-C16 isoform.

11. A method of producing at least one new mycosubtilin isoform selected from the group consisting of Gln1-C16 and Gln1-C17 isoforms comprising cultivating the strain as defined in claim 1.

12. A method of using the strain of claim 2 to produce a mycosubtilin isoform selected from the group consisting of Gln1-C16 and Gln1-C17 isoforms, the method comprising:
cultivating the strain.


SEQUENCE LISTING (26 sequences)

SEQ ID NO: 1 - DNA, 1770 bp, Bacillus subtilis
  source 1..1770: mol_type = genomic DNA
atgaaattttggaaaagcgtagtaggcaagctgtggtttacgatcctttcacttgtcttgatcgtattgttcattttaacggttcttttgctggaatttatcgagaattatcatgtggaagaagccgagaatgatttaacacagcttgcgaataaagttgctgtcatcttagaaaatcacgaggaccaggcactggcaagatcaatcacttgggagctcgctgataacttaacaagcatcgccattattcaggatgagaaaaagtactggtattctccgaatgataaaaatcggctgtcgtccattacggttgagcaaatacagcatgacaaagacttgaataaagctctccaagaccataaaaaagtaagcaaacgaacggggctgagcgatacagatacggataatgaacgactgattgtaggtgtcccgtatgaaaaagacggaaagaaaggcatggtctttttatctcagtctttgcttgccgttaaagatacaacaaaacacacgacccgctatattttcctggccgcgggaattgcgattgtactgaccacgttttttgctttcttcttatcaagcagggtcacgtatccgctccgaaaaatgagagaaggcgcacaggatttggcgaagggcaagttcgatacaaaaatcccgattttaacccaggatgaaattggtgaactagcaacagcttttaatcaaatgggccggcagcttaactttcatattaatgcgctcaatcaagaaaaagagcagctttctaacattttgagcagtatggctgacggggtcattaccattaatatcgacggcacgattcttgtgacaaaccctccggctgaacgttttcttcaggcttggtattatgagcagaatatgaatatcaaagaaggcgataaccttccgcctgaagcaaaagagctgttccaaaacgctgtcagcactgaaaaagagcaaatgattgagatgacgctgcaaggcagatcatgggtgctcttgatgtcgccgctttatgcggaatcgcatgtcagaggagcggttgccgtgctgcgcgacatgactgaagaacgccgtcttgataagctgcgggaggactttatcgcaaatgtcaaccatgagttgagaacaccgatctctatgcttcagggatacagtgaagcaattgtcgatgacattgcaagctccgaggaagaccggaaagaaattgctcaaatcatttatgacgaatcgttgcgaatgggccgtctagtcaatgatttgcttgatctagcccgaatggaatcaggccatacaggcttgcattatgaaaaagtcaatgtcaatgaatttttagagaaaatcattcgaaaattttccggtgttgctaaagaaaaaaatattactttacatcaagatatttctctcactgaagaagaatttatgtttgatgaagacaagatggagcaggtatttaccaacttgattgataacgcgctgcggcatacttcatccgaaggcagtgtttccattgcagtccattctgtgaaggatggattgaaaattgatatcaaagactctggatccggcattccggaagaagatctgccatttatctttgagcggttctataaagcagataaagcgcggacaagaggcagagcaggaaccggtcttgggctggctatcgttaaaaatatcgtggaagcccacaacggatcgattacggtgcacagtcgaatggataaaggaacgacatttacattttatattccgacaaaaccataa

SEQ ID NO: 2 - DNA, 3600 bp, Bacillus subtilis
  source 1..3600: mol_type = genomic DNA
ttgctagatgtgaacaattttgagtatatgaacatcggtcttgcttcaccggataaaatccgttcatggtcttttggtgaagtgaaaaagcctgaaacgataaactatcgtacgttaaaacctgaaaaggacggtctattctgcgaacgcattttcggaccgactaaggactgggaatgtcattgcgggaagtacaagcgagttcgttataaaggcgtagtttgtgaccgctgcggagtcgaagtaacacgggctaaagtccgtcgtgagagaatggggcacattgaactggctgccccagtttcccacatttggtatttcaaaggtattccaagccgtatgggtcttgtgctggatatgtcacctcgtgctttagaagaagtcatttactttgcttcttacgttgtaactgatccggcaaacacaccgcttgaaaagaaacagcttctgtctgaaaaagaatatcgtgcttatcttgataaatacggtaataagttccaagcgtctatgggtgcagaagcgattcataaacttcttcaagatatcgatcttgtaaaagaagttgatatgttaaaagaagagctgaaaacttcacaaggacagcgccgtacacgtgcgatcaaacgtcttgaagttttagaagccttccgtaactcaggaaacaagccttcttggatgatccttgatgtgcttcctgttattcctcctgagcttagaccgatggttcagctagatggcggacgttttgcgacttctgatttgaatgacctttatcgtcgtgtcatcaaccgtaacaatcgtttgaaacgccttttggaccttggtgcgcctagcatcattgttcaaaacgaaaagcgtatgcttcaagaggctgtcgatgctttaattgacaacggccgccgcggacgtcctgtaacaggtcctggtaacagaccgttaaaatctctttctcacatgttgaaagggaagcaaggccgtttccgtcaaaaccttcttggtaaacgtgtcgattactctggacgttctgtaatcgtcgttggtcctcatttgaaaatgtatcaatgcggattaccgaaggaaatggcgcttgaacttttcaaacctttcgttatgaaggagcttgttgaaaaaggtcttgctcacaacattaagagtgcgaaacgtaaaattgagcgcgtacagcctgaagtatgggatgtactggaatcagttattaaggagcatccggtacttctgaaccgtgcccctacacttcacagattaggtatccaagcgtttgaaccaacgctagttgaaggacgcgcaatccgtcttcacccgctcgtatgtacagcttacaacgctgactttgacggtgaccaaatggcggttcacgtaccattatctgctgaagctcaagctgaagcacgcatcttgatgcttgctgctcaaaacatcttgaaccctaaagatggtaaaccggttgtaacaccgtctcaggatatggtactgggtaactattacctgacacttgagcgtgccggtgctgtcggtgaaggtatggtcttcaagaatacagacgaagcgcttcttgcttatcaaaacgggtatgttcaccttcatacgagagtagctgttgcagctaactcccttaagaatgtgacatttaccgaagaacagcgttcaaaattgttaattacaactgtcggtaagcttgtcttcaatgaaattcttccggaatcattcccttacatgaatgaaccaacgaagagcaacattgaagagaaaacacctgaccgtttcttcttagaaaaaggtgccgatgttaaagctgttattgaacagcagccaatcaatgcaccatttaaaaaaggaattttgggtaaaatcatcgcggaaatctttaaacgattccatatcacggaaacgtctaaaatgcttgaccgcatgaaaaacctaggtttcaaatattcaacaaaagctggtattacagttggggtttctgacatcgtcgtactcgatgataaacaagaaattcttgaggaagcgcaaagcaaagttgataacgtcatgaagcaattccgacgtggtctaatcactgaagaagaacgctatgagagagttatctctatctggagtgctgcaaaagacgttatccaaggcaaactgatgaaatcacttgatgagctcaacccgatctacatgatgagtgactctggtgcccgtggtaacgcgtctaacttcacgcagcttgccggaatgcgcggcctgatggccaacccggctggacgtatcattgaattaccgatcaaatcaagtttccgtgaaggtctgacagtattggagtactttatctccactcacggtgcgcgtaaaggtcttgccgataccgcccttaaaactgctgactcaggttaccttacacgccgtctcgttgacgttgcgcaggatgttatcatccgtgaaactgattgcggaactgatcgaggaatccttgctaagcctcttaaagaaggaactgaaacaattgagcgcttagaagaacgcttaatcggccgttttgcacgaaaacaagtgaagcaccctgaaacaggtgaagtgcttgtaaacgaaaacgaactgatcgatgaagataaagcactggagattgtagaagccggtattgaagaagtgtggatccgttctgccttcacatgtaatacgcctcatggtgtatgtaaacgatgctatggccgaaatcttgcaactggctccgatgttgaagtcggtgaagctgtcggtatcattgctgcccaatcaatcggtgagcctggtacacagttaacaatgcgtacattccatacaggcggggttgctggagacgatatcactcagggtcttccgcgtatccaagagcttttcgaagcgcgtaatccgaaaggtcaggcaacaattacagaaatcgacggtacagtcgttgagatcaatgaagttcgtgataagcaacaggaaattgtggttcaaggcgcagtggaaacacgttcttatacggcaccttataactcccgcctgaaagtagcggaaggagataaaattactcgaggccaagtactgacagaaggttcaatcgatccgaaagagcttcttaaagtgactgacctgactactgttcaagagtatcttctccatgaagttcaaaaggtttaccgtatgcagggtgttgaaatcggcgataaacacgtagaggtaatggttcgccagatgcttcgcaaagtccgcgtaattgacgccggtgacactgatgtacttccaggtacattgcttgatattcaccaatttactgaagcgaacaaaaaggtattgcttgaaggcaatcgacctgctacaggccgtcctgtcttgctcggtattacaaaagcatctcttgaaactgattcattcttatctgctgcttccttccaggaaacaacacgtgtccttacagacgcagcgatcaaaggtaagcgtaatgagcttcttggcttgaaagagaatgttatcatcggtaagcttgttccagctggtacaggtatgatgaaataccgtaaagtaaaaccagtttcaaatgttcagccgactgaagacatggttccggttgagtaa

SEQ ID NO: 3 - DNA, 3501 bp, Bacillus subtilis
  source 1..3501: mol_type = genomic DNA
ttgggagcagagtttttagtaggcaggtccgggagtggaaaaacgaagctgatcatcgacagcattcaggatgaactgcgccgggctccattcgggaaaccgatcatttttctagtcccggatcaaatgacgtttttaatggaatacgagcttgctaaaacgcctgatatgggcggaatgatacgtgctcaagtgttcagcttttcacgattggcctggcgcgtcctccagcatacgggaggaatgagcaggccgtttcttacgagcacaggcgtacagatgctcctgcggaagctcattgaggagcataaacaggagtttaaagtctatcaaaaagcaagtgataaaagcggatttaccgcacaagttgagcggatgctgacagagtttaagcggtattgtctggaaccggaggatatccgccggatggcggaaagcggaacggcttccgagtatcgcggagaacgcgttttatctgaaaagctccatgacttatcgattctgtatcagcaaatggaaaaaagtcttgcagatcaatatcttcactctgaggattatttgacgttgctggcagagcatattccgcttgcggaagatataaaaggcgcgcatatctatgtggacggcttttatcagtttacgccgcaggaattcagggtgttggagcagcttatggttcatgcagagcacatcacgttttcgctgacagcggacaagccgtcttatgagcgagagccgcatgaactggaattgttcagaatgacggggaaaacgtattaccgcctgcatcagaaggcgaaggaactgaatttggacattacctataaagagctgagcgggattgagcggcatacgcagacgccggaattggcacatctagaggtgcagtatgaagcgcgcccggccatcccgtacgcagaaaaacaagaggcctttactgtgatgcaagccgcaaacagacgagctgagctggaaggcattgcccgggaaattcacactctagtcagagagaagggctatcgctataaagatgtagcgattcttgcgcgccagccggaagactacaaggatatggtgaaggaagttttcgcagattacgagattccttatttcattgacggaaaagcatctatgctgaaccatccgttaatcgaatttatccggtcgagcattgatgttctgaaagggaattggcgttatgaagcggtgtttcgctgcgtgaaaaccgaactgctattcccgcttaacgagccgaagactaaggtgagagaacaggtcgatcagcttgaaaattactgtatcgcctacgggatcaaaggcgatcgctggacaaagggcgaccgtttccattacagacgttttgtgtcattggatgatgattttgcgcaaaccgatcaggaaatcgaaatggaaaacatgctgaatgacacacgcgactggattgttccgccgctttttcagtttcaaaaacgaatgaaaaaagcaaagacggttcaggagaaggcggaggcgctctaccgctatctagaagagacggatgtaccgctgaagctggatcaggaaagacagcgtgctgaggaagacggcagaatcattgaagcgcagcagcatcagcaggcgtgggatgcagtcattcagctacttgaggagtttgttgaaatgatgggggatgatgagatttctcttgatttgtttcagcaaatgatggaagccggcgcggagtcgcttacattttctctaattccgcctgcgcttgaccaggtgtttgtcggcaatatggatttatccagaatgtacggcacctcttgcacctttgtgctgggggcaaacgacggcgttctgcctgcacgccctgatgaaaatggggtgctgtcggacgatgaccgggaatggctgaaaacgattgggattgagctttcctcaggcgggcgggagcgtctgcttgatgagcatttcctcatctatatggcgttctccagtccgtctgaccggctttacgtatcgtatccgattgctgatgcggaaggaaaaacgcttttgccgtcgattgtcattaaacggctggaagaactgtttccgcagcataaggagcggctgttgacaaatgaacctgaacaggtcagcgatgaggaacagcttatgtatgtcgtcaataaaagcgtggcacagtcctttaccgcaagccagctcagattatggactcgggaatacgacatcagcgacgtctggtggagcacgtacaatgtgctgatgagtgagccggacaggctgcaatcgaaaaagctgttctcaagcctgtttttccggaatgaagtgaagcagcttgaacgcagcgtgtcgagacagctctatggtgaacgcattcagggcagtgtatcgagaatggaaacctttaacgcgtgcccgttctcccatttcgcatcacacgggctgcatctaaaggaacggcaatttttcaagctcgaagcaccggatatcggccagctgtttcattccagcttaaagctgatttcagaccggctgcgcgagcaaaaaatgaattggcgcgatttaacgaaagagcagtgtgaactgttttcctatgatgcggtagagcggctggcaccgaaactgcaaaaggaaattttgctcagctcaaaccggcattattatgtgaaggaaaaactgcaaaaaattgtgacacgtgtgtccggtattttaagcgagcatgcgaaagcgagcgggtttgtgccgatcgggcttgaactgggctttggaggaaaagggccgcttccgccgctgacctttaagctgaaaaacggctgcacgatggaacttgtcgggcgaattgaccgtgttgataaagcagaaagctcaaaaggcctgcttctaaggattgtcgattataaatctagcgacaaaggccttgacttagcggaagtatattacggactggcgctgcaaatgctgacgtaccttgatttatcgattactcattcagccgactggctcggaatgagggcgacgccggcaggagtgctgtatttccatatacatgacccgatgattcaatctaatctcccgcttgggcttgacgagattgaacaggagatctttaagaaatttaaaatgaagggcttgctcctcggcgatcaggaagcggttcgccttatggatacgacccttcaagagggacggtcaaatatcatcaatgcgggcttgaaaaaagacggctctctcagatcggactcagcagcagtcggcgaaaaggaatttgagttgttgacgaaacatgtgcgccgcaccttccaagaagcaggcgaacaaatcaccgacggacgcgtatcgattgagccttataaaatgaagaacaagacgccgtgcacatactgtgcgttcaaatcagtatgccaatttgatgaatcattagaagagaacgaatatcgtccgttaaaggctgaaaaggataagacgatacttgagtggataaaaaaggaggcagatggcaatgaacattcctaa

SEQ ID NO: 4 - DNA, 1491 bp, Bacillus subtilis
  source 1..1491: mol_type = genomic DNA
atgcttcagacaaaggattatgaattctggtttgtaacaggaagccagcacctatacgggaaagagacgctggaactggtggatcagcatgcgaaaagcatttgtgaggggctcagcggggtttcttccagatataaaattacgcataagcccgtcgtcacttcatccgaaaccatcagacagctcttaagagaagcggagtacagtgagacatgcgccggcatcattacctggatgcacacattttctcccgcaaaaatgtggatagaaggcctttcttcttatcaaaaaccgcttatgcatctgcatactcaatataatcgcgatattccgtggggtacgattgacatggattttatgaacagcaaccaatccgcacacggcgatcgtgagtatggttacatcaactcgagaatggggctgagccgaaaaatcgttgccggctattgggatgatgaagaagtgaagaaagaaatatcccagtggatggatacggcggctgcattaaatgaaagcagacacattaaggtagccagattcggagataacatgcgtcacgtcgcggtaacagacggagacaaggtaggggcgcatattcaatttggctggcaagttgacggatatggcatcggagaccttgttgaagtgatgaaccgaattacggacgacgaggttgacacgctttatgcggagtatgacaggctttatgccatcagcgaggaaacaaaacgcgacgaagcaaaggtagcgtcgattaaagaacaggcgaaaattgaacttggcttaaccgcttttcttgaacaaggcggatactcagcttttacgacatcgtttgaagtgctgcacggaatgaaacagctgccgggacttgccgttcagcgcttgatggagaaaggctacgggttcgcaggtgaaggagattggaagacagcggctcttgtacggatgatgaaaatcatggctcaaggaaaaagaacgtccttcatggaagattatacgtatcattttgaaccgggaaatgaaatgattctgggctcgcacatgcttgaagtgtgtccgactgtcgctttggatcagccgaaaattgaggttcatccgctttctatcggcggaaaagaggaccctgcacgttttgtatttaatggcatcagcggttcagccattcaagcaagccttgttgatattggtgggcgtttccgtcttgttttgaatgaagtcaacggacaggaaattgaaaaagacatgccgaatctgccggtggcccgtgttctctggaagccggagccgtcattgaaaacagcggcggaggcatggattttagccggcggcgcacaccacacctgcttgtcttatgaactgacagtggagcaaatgctcgattgggcggaaatggcgggcatcgaaagtgttctcatttcccgtgatacgacaattcacaagctgaaacacgaattaaaatggaacgaggcgctttaccggcttcaaaagtag

SEQ ID NO: 5 - DNA, 489 bp, Bacillus subtilis
  source 1..489: mol_type = genomic DNA
atgagctgcggaaaaacccatggccggcatgagaactgtgtatgcgatgcagtggaaaagattttagcagagcaggaggcagtggaagaacagtgtccgactggctgctatacaaaccttttgaatccgacaattgcaggtaaagacacaatttcgttcctcttgtttgataaaaaaggcggattgttctccacattcggaaacgtggggggatttgtggatgatatgcaatgctttgagtccattttcttccgcgttgaaaaattatgcgattgctgcgcaacactgtctattttacgcccggtcgatgtcaaatgcgatactttaagtgtttgccacccttgtgacccggatttcttcgggctagaaaaaacagatttttgcatcgaagtggatctcggatgcttctgtgcgattcaatgcctgtcaccagagttagttgacagaacttcgcctcataaggataaaaagcatcatcacaatggataa

SEQ ID NO: 6 - DNA, 330 bp, Bacillus subtilis
  source 1..330: mol_type = genomic DNA
ttggcaaatgaattgaatatttctggagtttttccaattttgttacagcatatcaagaaatggcgtctttatcgagaaacagatagagatgccatttatacgattgagttagcagctgtttatcatttccgtccaattatttttacattcaaagacgtttcagccgttacgatacgagatttggaaaccctttcggatatgtctgattgccttctccgcattgaattgcagccgcaagggaggtaccttgtggaagaatgggaggaggagacgtttcgcttttattgccggaaatatgagatgacagaagagagaggatcacgtgtttga

SEQ ID NO: 7 - DNA, 387 bp, Bacillus subtilis
  source 1..387: mol_type = genomic DNA
atgatatataaagcatttgttaaaggacttgatccacatatagaagaggaagtgattctcgaaatagaagggatagaatttacaggttttttttctgtctgtccttatgaaattgaagtggggaaaaaatatcctgtgttagttggttttacgattttggatgagttagttattcgtgaaaataaagaaaaaacaaaagaattagatcggattggtttaggatatcaatattatattaggggaattttgcaagaggattcaattgatgcaggaatcgttctaactgatgaagatgagtatttttctgaatatcctcatttaattgggaaaaacgttgagatgaaggttgacaggataagcatagaatttttaagagagtctgtttaa

SEQ ID NO: 8 - protein, 7 aa, Bacillus subtilis
  MOD_RES 3: note = D-asparagine
  MOD_RES 2: note = D-tyrosine
  MOD_RES 6: note = D-serine
  CROSSLNK 1..7: note = head-to-tail peptide bond between Asn-1 and Asn-7
NYNQPSN

SEQ ID NO: 9 - protein, 7 aa, Bacillus subtilis
  MOD_RES 3: note = D-glutamine
  MOD_RES 6: note = D-serine
  MOD_RES 2: note = D-tyrosine
  CROSSLNK 1..7: note = head-to-tail peptide bond between Asn-1 and Asn-7
NYQQPSN

SEQ ID NO: 10 - protein, 7 aa, Bacillus subtilis
  CROSSLNK 1..7: note = head-to-tail peptide bond between Asn-1 and Asn-7
  MOD_RES 3: note = D-asparagine
  MOD_RES 6: note = D-serine
  MOD_RES 2: note = D-tyrosine
QYNQPSN

SEQ ID NO: 11 - protein, 7 aa, Bacillus subtilis
  CROSSLNK 1..7: note = head-to-tail peptide bond between Gln-1 and Asn-7
  MOD_RES 3: note = D-asparagine
  MOD_RES 2: note = D-tyrosine
  MOD_RES 6: note = D-serine
QYNQPSN

SEQ ID NO: 12 - protein, 7 aa, Bacillus subtilis
  CROSSLNK 1..7: note = head-to-tail peptide bond between Asn-1 and Asn-7
NYNQPSN

SEQ ID NO: 13 - protein, 7 aa, Bacillus subtilis
  CROSSLNK 1..7: note = head-to-tail peptide bond between Asn-1 and Asn-7
NYQQPSN

SEQ ID NO: 14 - protein, 7 aa, Bacillus subtilis
  CROSSLNK 1..7: note = head-to-tail peptide bond between Asn-1 and Asn-7
NYNQPSN

SEQ ID NO: 15 - protein, 7 aa, Bacillus subtilis
  CROSSLNK 1..7: note = head-to-tail peptide bond between Asn-1 and Gln-7
NYNQPSQ

SEQ ID NO: 16 - protein, 7 aa, Bacillus subtilis
  CROSSLNK 1..7: note = head-to-tail peptide bond between Gln-1 and Asn-7
QYQQPSN

SEQ ID NO: 17 - protein, 7 aa, Bacillus subtilis
  CROSSLNK 1..7: note = head-to-tail peptide bond between Asn-1 and Gln-7
NYQQPSQ

SEQ ID NO: 18 - protein, 7 aa, Bacillus subtilis
  CROSSLNK 1..7: note = head-to-tail peptide bond between Gln-1 and Gln-7
QYQQPSQ

SEQ ID NO: 19 - protein, 7 aa, Bacillus subtilis
  CROSSLNK 1..7: note = head-to-tail peptide bond between Asn-1 and Asn-7
NYQQPSN

SEQ ID NO: 20 - protein, 7 aa, Bacillus subtilis
  CROSSLNK 1..7: note = head-to-tail peptide bond between Gln-1 and Gln-7
QYNQPSQ

SEQ ID NO: 21 - protein, 7 aa, Bacillus subtilis
  CROSSLNK 1..7: note = head-to-tail peptide bond between Gln-1 and Asn-7
QYQQPSN

SEQ ID NO: 22 - protein, 7 aa, Bacillus subtilis
  CROSSLNK 1..7: note = head-to-tail peptide bond between Asn-1 and Gln-7
NYQQPSQ

SEQ ID NO: 23 - protein, 7 aa, Bacillus subtilis
  CROSSLNK 1..7: note = head-to-tail peptide bond between Asn-1 and Asn-7
NYQQPSN

SEQ ID NO: 24 - protein, 7 aa, Bacillus subtilis
  CROSSLNK 1..7: note = head-to-tail peptide bond between Asn-1 and Gln-7
NYNQPSQ

SEQ ID NO: 25 - protein, 7 aa, Bacillus subtilis
  CROSSLNK 1..7: note = head-to-tail peptide bond between Gln-1 and Asn-7
QYNQPSN

SEQ ID NO: 26 - protein, 7 aa, Bacillus subtilis
  CROSSLNK 1..7: note = head-to-tail peptide bond between Asn-1 and Asn-7
NYNQPSN